(19) United States
(12) Patent Application Publication
(10) Pub. No.: US 2020/0009056 A1
(43) Pub. Date: Jan. 9, 2020

(54) ORGANIC COMPOUNDS

(71) Applicant: Novartis AG, Basel (CH)

(72) Inventors: Holger Petersen, Weil an Rhein (DE); Olivier Jean Lambert, Steinbrunn le haut (FR); Rolf Löffler, Freiburg (DE); Michael Ausborn, Lorrach (DE); Jean-Daniel Bonny, Fullinsdorf BL (CH)

(21) Appl. No.: 16/576,217

(22) Filed: Sep. 19, 2019

Related U.S. Application Data
(63) Continuation of application No. 15/627,619, filed on Jun. 20, 2017, now abandoned.
Application No. 15/627,619 is a continuation of application No. 14/330,485, filed on Jul. 14, 2014, now abandoned.
Application No. 14/330,485 is a continuation of application No. 11/650,701, filed on Jan. 8, 2007, now abandoned.
Application No. 11/650,701 is a continuation of application No. 10/313,709, filed on Dec. 6, 2002, now abandoned.

(30) Foreign Application Priority Data
Nov. 19, 2002 (GB) 0226993.4
Nov. 29, 2002 (GB) 0227883.6

(51) Int. Cl. A61K 9/16; A61K 9/00; A61K 47/10; A61K 47/26; A61K 47/38; A61K 38/08

(52) U.S. Cl. CPC A61K 9/1647; A61K 9/0019; A61K 38/08; A61K 47/26; A61K 47/38; A61K 47/10

(57) ABSTRACT

A pharmaceutical composition comprises octreotide acetate microparticles of linear poly (lactide-co-glycolide) polymer wherein the polymer contains less than 1% silicone oil or heptane.

The present invention relates to pharmaceutical compositions, in particular to depot microparticles.

Octreotide acetate microparticles for injectable suspension are commercialized as pharmaceutical compositions under the brand name SANDOSTATIN LAR. These pharmaceutical compositions are indicated for inter alia long-term maintenance therapy in acromegalic patients, and treatment of severe diarrhea and flushing associated with malignant carcinoid tumors and vasoactive intestinal peptide tumors (vipoma tumors). The pharmaceutical compositions are normally administered once-a-month. The octreotide is presented as a sterile pharmaceutical composition in a vial which when mixed with a vehicle for suspension such as sterile water becomes a suspension that is administered by an intragluteal injection.

The octreotide acetate microparticles are produced from the acetate salt of octreotide which is distributed throughout a biodegradable poly (DL-lactide-co-glycolide)-glucose star polymer (disclosed in e.g. U.S. Pat. No. 5,922,682, the contents of which are incorporated herein by reference). The octreotide acetate microparticles are produced according to the teaching of U.S. Pat. No. 5,538,739 (the contents of which are incorporated herein by reference) involving use of silicone oil and heptane. Traces of these starting materials may be detected in the final product.

Until now, no octreotide composition based on linear poly (lactide-co-glycolide) in sustained release form for parenteral administration has reached the market.

The present invention provides commercially acceptable octreotide acetate microparticles produced from linear poly (lactide-co-glycolide) (hereinafter referred to as PLG) which have similar pharmacokinetic characteristics to SANDOSTATIN LAR with acceptable drug loading whilst also of high purity and which may be free from silicone oil and heptane.

Accordingly in one aspect the present invention provides

i) a pharmaceutical composition comprising octreotide acetate microparticles of linear poly (lactide-co-glycolide) polymer wherein the polymer contains less than 1% wt./wt. silicone oil or heptane or

ii) a pharmaceutical composition comprising octreotide acetate microparticles of linear poly (lactide-co-glycolide) polymer wherein the polymer is free from silicone oil or heptane (both of which are hereinafter referred to microparticles of the invention).

In another aspect the present invention provides a process for the production of octreotide acetate microparticles, comprising the steps of:

a) dissolving or dispersing octreotide acetate in methylene dichloride containing a dissolved linear poly (lactide-co-glycolide) to form a dispersion or a homogeneous solution;

b) combining said dispersion with an effective amount of a continuous process medium to form an emulsion that contains said process medium and microdroplets comprising said octreotide acetate, said solvent and said linear poly (lactide-co-glycolide); and

c) immediately after the formation of said emulsion, adding all at once said emulsion to an effective amount of an extraction medium to extract said solvent from said microdroplets to form said microparticles.

The process may be effected in conventional manner. Thus, high speed stirrers may be used to produce emulsions.

In step a) the PLO is preferably dissolved in methylene dichloride at a concentration of from about 1% to about 40%, typically 2-2.5%. Octreotide acetate is preferably dissolved in a polar organic solvent miscible with methylene dichloride, preferably methanol.

An aqueous solution of octreotide acetate may be used, preferably a water solution, which is then emulsified with the polymer solution to form an emulsion.

The concentration of octreotide acetate in polar organic solvent or in aqueous solution is preferably from about 1% to about 20%, preferably 4 to 10%, very preferably 4 to 7%, most preferably 5%. It is preferred to have a homogeneous solution after mixing of the octreotide with the dissolved linear poly (lactide-co-glycolide). Mixture of up to about 20% v/v of (i) a solution containing methanol with (ii) methylene dichloride containing a dissolved linear poly (lactide-co-glycolide) may still result in a homogeneous solution, e.g. up to about 20% of solution (i) in a mixture of (i)+(ii). The weight ratio of component (i) to component (ii) is typically about 1:8. In step b) an emulsion may be produced by dispersing the octreotide acetate/PLG—methylene dichloride mixture into an aqueous processing medium (the continuous phase which is preferably saturated with the polymer solvent, methylene dichloride).

Prior to the addition of the mixture containing the PLG/octreotide, the process medium is preferably saturated with methylene dichloride to reduce extraction of solvent from the microdroplets during formation of the emulsion. The process medium is then mechanically agitated with devices such as homogenizers, propellers or the like, as the PLG/octreotide mixture is added to the process medium. During this step of the process, no solvent may be generally evaporated or removed from the microdroplets. The temperature at which the emulsion is formed is not particularly critical, except that it may be within a range that will prevent the methylene dichloride from boiling or the process medium from gelling or freezing or the octreotide or PLG from degrading. The time required to form an emulsion is quite short. Generally, emulsions may be formed within 30 seconds to 5 minutes, depending upon the surfactant used and the method of agitation of the process medium.

Preferably a stabilizing agent for emulsions produced in microparticle processes is present to prevent agglomeration. The concentration present may affect the final size of the microparticles. Generally the concentration of the emulsion stabilizing excipient in the process medium will be from 0.01% to about 20% depending on the surfactant, the polymer solvent, and the processing medium used. The amount of stabilizing agent is preferably from about 0.025 to about 1%.

Suitable stabilizing agents include:

A) polyvinyl pyrolidone: Suitably the molecular weight may vary between 2000 and 20000 daltons. Suitable examples include these commonly known as Povidone K12 F (average molecular weight about 2500 daltons), Povidone K15 (average molecular weight about 8000 daltons) or Povidone K17 (average molecular weight about 10000 daltons). Preferably, the polyvinyl pyrolidone is present is present in an amount of from about 0.1 to about 20%, e.g. 5%.

B) carboxymethyl cellulose sodium: Preferably it has a low molecular weight. The viscosity may be, e.g. up to 20 cP for a 2% aqueous solution or a viscosity of from 8 to 25 mPa s. Conveniently the degree of substitution is from about 1.15 to about 1.45. Typically the sodium content is about 10.5% to about 12%.

C) polyvinyl alcohol (PVA): In one embodiment, the polyvinyl alcohol has a molecular weight from about 10000 to about 90000 daltons, e.g. about 30000 daltons.

Conveniently the polyvinyl alcohol has low viscosity having a dynamic viscosity of from about 3 to about 9 mPa s when measured as a 4% aqueous solution at 20° C. or by DIN 53015. Suitably the polyvinyl alcohol may be obtained from hydrolyzing polyvinyl acetate. Preferably, the content of the polyvinyl acetate is from about 10 to about 90% of the polyvinyl alcohol. Conveniently the degree of hydrolysis is about 85 to about 89%. Typically the residual acetyl content is about 10 to 12%. Preferred brands include Mowiol 4-88 and 8-88 available from Clariant AG Switzerland.

Preferably the polyvinyl alcohol is present in an amount of from about 0.1 to about 5%, e.g. 0.5%.

D) gelatin: Preferably the gelatin is porcine or fish gelatin. Conveniently the gelatin has a viscosity of about 25 to about 35 cps for a 10% solution at 20° C.

Typically pH of a 10% solution is from about 6 to about 7. A suitable brand has a high molecular weight, e.g. Norland high molecular weight fish gelatin obtainable from Norland Products Inc, Cranbury N.J. USA. Preferably the gelatin is present in an amount of from about 0.01 to about 5%, e.g. 0.05%.

Conveniently polyvinyl alcohol may be used.

In step c) transferring all of the emulsion immediately to a large volume of processing medium or other suitable extraction medium to immediately extract the solvent from the microdroplets in the emulsion forms microparticles of the invention.

As soon as an emulsion forms, all of the process medium containing the organic microdroplets is transferred, as quickly as possible, to an extraction medium so that greater than 20% to 30% of the solvent may be immediately removed from the microdroplets (e.g., within 3 minutes). Normally, water is used as the extraction medium but other solvents or oils can also be used. In addition, salts may be added to the extraction medium to adjust its ionic strength or pH. The amount of extraction medium used may be somewhat critical in that sufficient medium must be present to allow approximately immediate extraction of the solvent out of the microdroplets. Accordingly, the volume of the extraction medium will depend on the solvent used to dissolve the wall material and its solubility in the extraction medium. Generally, the volume of the extraction medium should be at least the volume needed to dissolve all of the solvent out of the microdroplets, preferably a volume 10-fold or higher.

In one embodiment, the added water is at a pH of about 7 or higher. Such pH may be adjusted to increase the encapsulation efficiency of the octreotide in the microparticles of the invention.

Preferably an aqueous sodium dihydrogen phosphate/sodium hydrogen phosphate buffer solution is present

After extraction of all or almost all of the solvent from the microdroplets (generally within 15 to 30 minutes), the hardened microparticles may be collected by centrifugation, filtration, or the like.

In another aspect the present invention provides

In a process for the production of octreotide acetate microparticles by an emulsion process, the improvement which comprises the steps of:

a) mixing octreotide acetate in methanol with methylene dichloride containing a dissolved linear poly (lactide-co-glycolide) to form a solution; and

b) emulsifying said solution with water.

The process may be effected as described above.

In one embodiment gelatin is not used and is absent in the microparticles of the invention.

The lactide may be D, L or mixtures thereof e.g. racemic DL lactide.

Homopolymers, e.g. poly(DL-lactide) homopolymers, may be used. The molecular weight of the homopolymers is from about 7,000 to 25,000 daltons, e.g. 18 000 daltons.

Preferably the polymer used is poly (DL-lactide-glycolide).

The ratio of lactide to glycolide units in the PLG may vary between wide limits. It is however preferred to have a molar ratio of from 90 to 10 to 40:60 lactide to glycolide units, e.g. (i) 50:50 poly (lactide-glycolide) or (ii) 75:25 poly (lactide-glycolide) or (iii) 65:35 poly (lactide-glycolide).

The polymers may be pure poly(lactide-glycolide) polymers or copolymers with other units. Preferably they are pure poly(lactide-glycolide) polymers.

Typically the average molecular weight of the PLG is from about 5,000 to about 70,000 daltons, e.g. 13 000, preferably it is from about 30,000 to about 70,000 daltons, especially from about 40,000 to about 60,000 daltons, more especially about 50,000 daltons.

The inherent viscosity of the PLO may vary between wide limits. It is however preferred to be in the range from about 0.1 to about 0.8 dl/g, e.g. from about 0.2 to about 0.8 dl/g in hexafluroisopropanol or preferably chloroform when measured under standard conditions, e.g. 20° C. A preferred example has a viscosity of from 0.45-0.55 dl/g in chloroform.

Preferably the polymer is amorphous. The linear polymer of the invention is not a star polymer and contains less than 5%, or is preferably is free from, star polymers e.g. a reaction product of a polyol containing at least 3 hydroxyl groups and having a molecular weight of up to 20,000 or a reactive derivative thereof and lactic acid or a reactive derivative thereof and glycolic acid or a functional derivative thereof. These products are disclosed in U.S. Pat. No. 5,922,682.

The linear polymers of the invention may be produced in conventional manner e.g. conventional techniques such as polycondensation and ring-opening of dimers. The production may be according to the teachings of U.S. Pat. No. 3,773,919 (the contents of which are hereby incorporated herein by reference). The polymer may be a reaction product of lactic acid or a reactive derivative thereof e.g. D,L-lactide, and glycolic acid or a functional derivative thereof, e.g. glycolide. There may be present a suitable catalyst for the production of linear polymers for example zinc oxide, zinc carbonate, basic zinc carbonate, diethyl zinc, organotin compounds, for example stannous octoate (stannous 2-ethylhexanoate), tributylaluminium, titanium, magnesium or barium compounds or litharge Stannous octoate (stannous 2-ethylhexanoate) is preferred.

The polymer is preferably obtained from Birmingham Polymers Inc. Birmingham, Ala., USA.

The octreotide acetate may be produced in conventional manner, e.g. as disclosed in U.S. Pat. No. 4,395,403, the contents of which are incorporated herein by reference.

Insofar as any aspect of production of the microparticles of the invention are not disclosed herein, such production aspect may be effected in conventional manner or in a manner analogous to known methods.

The amount of octreotide on or near the surface and hence the initial drug burst may be reduced by briefly washing the microparticles of the invention with water, e.g. including a 1/15 molar acetate buffer at pH 4.0 during 5 minutes.

The microparticles of the invention may be dried, e.g. to remove water and other volatiles like methylene dichloride.

In the drying step, the microparticles of the invention may be subjected to:

1) a freeze-drying process or

2) vacuum of 50 to 10⁻² millibars, e.g. 30 millibars or

3) addition of mannitol in powder to the filtered microparticles in a tumbler, under vacuum as in the drying process 2) and heating of 45° C. to 55° C., preferably 48 to 54° C., most preferably 50 to 52° C.

The volatile solvent, e.g. methylene dichloride, can alternatively be removed from the microparticles in suspension in aqueous solution, preferably a buffered water solution, e.g. potassium/sodium phosphate optionally, under vacuum conditions of the drying process 2). Preferably the microparticles of the invention may be purged with nitrogen or another inert gas. If desired the microparticles of the invention may be heated, e.g. from 25 to 55° C., preferably from 48 to 54° C. Duration of the drying period may be, e.g. from 2 hours to 5 days.

Hence, the present invention provides a process for the production of octreotide acetate microparticles by an emulsion process, which comprises the step of removing volatile solvents, e.g. methylene dichloride.

The resultant microparticles may be free-flowing powders of spherical particles.

The microparticles of the invention contain preferably less than 1% silicone oil, e.g. less than 0.5 or 0.1%, preferably less than 0.05%, especially less than 0.01%, silicone oil.

The microparticles of the invention include for example less than 0.5%, e.g. less than 0.2%, preferably less than 0.1%, methylene dichloride.

The microparticles of the invention include for example less than 0.05%, e.g. less than 0.03%, preferably less than 0.01% especially less than 0.005% or 0.001%, methanol.

The microparticles of the invention include for example less than 3%, e.g. less than 1%, less than 0.1% preferably less than 0.05 or 0.01%, especially less than 0.005%, polyvinyl alcohol.

The microparticles of the invention include for example less than 2%, e.g. less than 1 or 2.%, preferably less than 0.1% heptane, especially less than 0.01% or 0.005% heptane.

The microparticles of this invention may have e.g. a size range from about 1 to 250 preferably 10 to 200, especially 10 to 130 microns in diameter. Mean diameters may be e.g. from 80 to 100 microns.

The size distribution of the microparticles of the invention has preferably at least one of the following average diameter characteristics:
99% or more smaller than 130 microns
90% or more smaller than 90 microns
80% or more smaller than 70 microns
95% or more greater than 10 microns

based on the average size distribution as measured by conventional light scattering methods.

It is preferred to have a broad size distribution of the microparticles of the invention.

The microparticles of the invention may exhibit a smooth to rough surface.

It is preferred to have a smooth surface in the microparticles of the invention. The smoothness may be determined in conventional manner, e.g. by visual determination by electron microscopy.

The microparticles of the current invention are usually made up of particles of a spherical shape, although microparticles may be irregularly shaped.

Preferably the surface area varies by about 5% from the corresponding surface area of a sphere.

Additionally the content uniformity of a unit dose is excellent. Unit doses may be produced which vary from about 85 to about 115%, e.g. from about 90 to about 110%, or from about 95 to about 105%, of the theoretical dose.

Preferably the microparticles of the invention are dense rather then porous. The porosity may be determined in conventional manner, e.g. by visual determination by BET-nitrogen-sorption/Hg-porosimetry or electron microscopy, e.g. by observing the diameter and extent of channels in a cut microparticle.

Preferably the microparticles of the invention contain less than 4%, especially less than 3% and preferably less than 2% total octreotide degradation products.

The present invention also provides a pharmaceutical composition comprising microparticles of the invention.

The pharmaceutical composition may be in the dry state. Preferably the pharmaceutical composition contains a vehicle to facilitate reconstitution. The vehicle preferably comprises from about 1% to about 40% of the pharmaceutical composition.

The microparticles of the invention may comprise from about at least 90% of the pharmaceutical composition.

The vehicle may contain excipients e.g. an anti-agglomerating agent, a viscosity-increasing agent or an isotonizing agent.

A suitable anti-agglomerating agent includes mannitol. Mannitol may also serve as a suitable isotonizing agent. Preferably, this is present in about 2-5%, e.g. 4% of the pharmaceutical composition or in about 0.1 to 1%, e.g. 0.5 to 0.8% of the pharmaceutical composition.

A suitable viscosity-increasing agent includes carboxymethyl cellulose sodium. Preferably carboxymethyl cellulose sodium has a low viscosity. Embodiments may be as described above. Typically it has a high molecular weight. The viscosity may be, e.g. from 10 to about 15 mPa s when measured as a 1% (w/v) aqueous solution at 25° C. in a Brookfield LVT viscometer with a spindle 1 at 60 rpm.

Conveniently the degree of substitution is from about 1.15 to about 1.45. Typically the sodium content is about 10.5% to about 12%. Preferably this is present in about 0.1-1% e.g. 0.5% of the pharmaceutical composition.

Preferably a wetting agent is present. Such wetting agents preferably include non-ionic surfactants.

a) Poloxamers also known as polyoxyethylene polyoxypropylene block copolymers Preferably this is solid.

In one embodiment the molecular weight is from about 2000 to about 8000 daltons. The degree of polymerization of the ethylene moiety is typically 80 to about 110 units. The degree of polymerization of the propylene moiety is typically 20 to about 60 units. Examples of such compounds suitable for use in accordance with the present invention are those known and commercially available, e.g. under the trade name Pluronic F 68 available from BASF Germany.

b) Polyoxyethylene-sorbitan-fatty acid esters e.g. mono- and trilauryl, palmityl, stearyl and oleyl esters e.g. of the type known and commercially available under the trade name TWEEN

20 [polyoxyethylene(20)sorbitanmonolaurate],

40 [polyoxyethylene(20)sorbitanmonopalmitate],

60 [polyoxyethylene(20)sorbitanmonostearate],

80 [polyoxyethylene(20)sorbitanmonooleate],

65 [polyoxyethylene(20)sorbitantristearate],

85 [polyoxyethylene(20)sorbitantrioleate],

21 [polyoxyethylene(4)sorbitanmonolaurate],

61 [polyoxyethylene(4)sorbitanmonostearate], and

81 [polyoxyethylene(5)sorbitanmonooleate].

Especially preferred products of this class for use in the pharmaceutical compositions of the invention are the above products TWEEN 40 and TWEEN 80.

Such wetting agents are preferably present in about 0.01 to about 0.1% of the pharmaceutical composition.

If desired the pharmaceutical composition in the dry state may comprise an anti-agglomerating agent such as mannitol.

The pharmaceutical composition may be stored under aseptic conditions e.g. in a vial. All steps are conveniently effected under sterile conditions using sterile material, e.g. produced using sterile filters.

Microparticles of the present invention may be stored in the form of a powder. For administration as injection the microparticles are suspended in a suitable vehicle for suspension.

A vehicle for suspension may comprise a viscosity increasing agent and/or wetting agent as mentioned above and additionally water.

In the further aspect the invention provides a pharmaceutical composition comprising octreotide acetate microparticles of linear poly (lactide-co-glycolide) polymer admixed or in association with a non-ionic surfactant (herein these compositions are also referred to as compositions of the invention).

The amount of liquid vehicle for suspension is preferably about 1 to 5 ml, e.g. 2-2.5 ml per dose. The liquid may be mixed with the dry pharmaceutical composition just prior to administration.

If desired a dry pharmaceutical composition and an aqueous vehicle for reconstitution may be housed separately in a double chamber syringe.

Excipients disclosed in the literature, as for instance in the components of the compositions of the invention may be described in Fiedler, H. P. “Lexikon der Hilfsstoffe für Pharmazie, Kosmetik and angrenzende Gebiete”, Editia Cantor Verlag Aulendorf, Aulendorf, 4^{th} revised and expanded edition (1996) Germany and “Handbook of Pharmaceutical Excipients”, Edited by A. H. Kibbe, American Pharmaceutical Association, Third Edition (2000), as well as manufacturer's brochures, the contents of which are incorporated herein by reference, may be used in the pharmaceutical compositions according to the invention.

All percentages as used herein are w/w except where otherwise specified.

The pharmaceutical compositions of the invention may be administered by an intragluteal, intramuscular or subcutaneous injection. The pharmaceutical compositions of the invention administered by injection provide an effective treatment of diseases over an extended period, e.g., over 2 to 6 weeks. The microparticles allow a controlled release of octreotide by diffusion and therefore steady-state levels of the drug are obtained over the extended period. The microparticles of the invention may be used for the same indications as known octreotide acetate microparticles.

The exact dose of octreotide will depend on a number of factors, including the condition to be treated, the severity of the condition to be treated, the weight of the subject and the duration of therapy.

The exact dose of microparticles of the invention used will depend on a number of factors, including the rate of release of octreotide and the desired duration of treatment.

The amounts may be determined using standard animal and clinical tests, e.g. bioavailability tests using rabbits, using SANDOSTATIN LAR as a standard. Octreotide levels may be determined using conventional methods e.g. gas chromatography or high performance liquid chromatography. Typically for SANDOSTATIN LAR in humans an initial drug burst is seen, declining to a nadir in the next few days, followed by a plateau phase for 2 to 3 weeks post injection. For example at the 20 mg octreotide dose maximum serum concentrations of about 800 ng/l may be reached from day 21 and last for 4 weeks.

If desired conventional in vitro tests may be used. In one such test SANDOSTATIN LAR in acetate buffer pH 4 at 0.1 mM a continuous release profile is observed. Preferably pure water is used. Release characteristics of octreotide may be any of the following:

Not more than 1.5% of octreotide dose in one hour, e.g. at least 0.2%

Not more than 4% of octreotide dose in 4 hours; or

Not more than 7% of octreotide dose in 24 hours.

It is preferred to use single administration of the microparticles of the invention ever 30 days. The pharmaceutical compositions of the invention preferably include 10, 20 or 30 mg of octreotide.

Conveniently the loading of octreotide in the microparticles of the invention is from about 1 to about 7%, e.g. from about 3 to about 7%, typically 4 to 6%, e.g. 5%.

The present invention also provides:
a) Use of the octreotide for the manufacture of microparticles or pharmaceutical compositions of the invention to be administered to a patient for the treatment of a disease treatable by octreotide, e.g. acromegaly.
b) A method of administering of the octreotide e.g. for the treatment of acromegaly, said method comprising administering to a patient in need of octreotide therapy microparticles or a pharmaceutical composition of the invention.

Following is a description by way of example only of depot formulations of this invention.

### EXAMPLE 1: MICROPARTICLES

Step a): Approximately 2.5 g of poly(DL-lactide-co-glycolide) [polymer] are dissolved in 25 g methylene chloride to prepare a 9 wt. % polymer solution. After the polymer is completely dissolved, 188 mg octreotide acetate [drug] in 3.7 g methanol are added and allowed to dissolve.

Step b): This polymer/drug solution is then poured into a 1-L vessel containing 400 g of 5.0 wt. % polyvinyl alcohol (PVA). The PVA is stirred at about 750 rpm by a 2.5 inch impeller (e.g. TEFLON driven by a Fisher Stedi-speed motor). The PVA is also saturated with 7 ml of methylene chloride prior to the addition of the polymer/drug solution. The resulting emulsion is allowed to stir for 7 min.

Step c): The vessel contents are transferred all at once to 12.0 litres of stirred deionized water. The microparticles are stirred in the deionized water for approximately 30 min and then were collected over 45-μm mesh size meter and 212-μm mesh size stainless steel mesh steel sieves arranged in series. The microparticles are rinsed with additional deionized water and allowed to air dry.

The microparticles of the invention obtained have the characteristics described above.

### EXAMPLE 2

Vehicle Composition

The microparticles of example 1 are mixed with mannitol and aseptically filled into two chamber syringe (TCS) consisting of one compartment containing the microparticles and one compartment containing a vehicle for suspension of the microparticles.

### Vehicle Composition

| | mg/ml |
|---|---|
| Pluronic F68 | 2.0 |
| Sodium-carboxymethylcellulose | 10.0 |
| (Blanose 7LFD) | |
| Mannitol | 6.0 |
| Water for injections | ad 2.0 ml |
| Nitrogen | q.s. |

The components are for a 10 mg dose of octreotide in the microparticles of the invention for a dry pharmaceutical composition. 2 ml of water are provided.

1. A process for the production of octreotide acetate microparticles, comprising the steps of:
a) forming a solution or dispersion by combining a first mixture which comprises octreotide acetate dissolved in a polar organic solvent that is miscible with methylene dichloride, with a second mixture which comprises a linear poly (lactide-co-glycolide) free from silicone oil or heptane dissolved in methylene chloride;
b) combining said solution or dispersion with an effective amount of an aqueous process medium to form an emulsion that contains said process medium and microdroplets which are free from silicone oil or heptane comprising said octreotide acetate, said methylene dichloride and said linear poly (lactide-co-glycolide); and
c) forming said microparticles by combining said emulsion with an effective amount of an extraction medium.

2. A process of claim 1 wherein said polar organic solvent that is miscible with methylene dichloride is methanol.

3. A process of claim 1 wherein said second mixture contains from about 1% to about 40% of linear poly (lactide-co-glycolide).

4. A process of claim 3 wherein said second mixture contains from 2% to 2.5% of linear poly (lactide-co-glycolide).

5. A process of claim 1 wherein said first mixture contains from about 1% to about 20% of octreotide acetate.

6. A process of claim 5 wherein said first mixture contains from 4% to 10% of octreotide acetate.

7. A process of claim 6 wherein said polar organic solvent that is miscible with methylene dichloride is methanol.

8. A process of claim 1 wherein said aqueous process medium is water saturated with methylene dichloride.

9. A process of claim 8 wherein said aqueous process medium comprises a stabilizing agent.

10. A process of claim 9 wherein said stabilizing agent is selected from the group consisting of polyvinyl pyrolidone, carboxymethyl cellulose sodium, polyvinyl alcohol and gelatin.

11. A process of claim 9 wherein said stabilizing agent is polyvinyl alcohol.

12. A process of claim 1 wherein said extraction medium is aqueous.

13. A process of claim 12 wherein said extraction medium is an aqueous sodium dihydrogen phosphate/disodium hydrogen phosphate buffer solution.

14. A process of claim 12 wherein said extraction medium has a pH of 7 or higher.

15. A process of claim 12 wherein said extraction medium is distilled water.

16. A process of claim 1 wherein said linear poly (lactide-co-glycolide) has a molecular weight in the range from about 30,000 to 70,000 daltons.

17. A pharmaceutical composition which comprises microparticles obtained according to the process of claim 1.

18. Microparticles obtainable according to the process of claim 1.

19. Microparticles of claim 18 which are free from silicone oil or heptane.
